(19) United States
(12) Patent Application Publication
(10) Pub. No.: US 2022/0265456 A1
(43) Pub. Date: Aug. 25, 2022

(54) DUAL-ARM SUPPORT SLING

(71) Applicant: ARMBIE LLC, Tulsa, OK (US)

(72) Inventor: Christian OSSE, Tulsa, OK (US)

(73) Assignee: ARMBIE LLC, Tulsa, OK (US)

(21) Appl. No.: 17/675,815

(22) Filed: Feb. 18, 2022

Related U.S. Application Data
(63) Continuation-in-part of application No. 29/771,100, filed on Feb. 19, 2021.

(51) Int. Cl. A61F 5/37; A41D 27/20

(52) U.S. Cl. CPC A61F 5/3738; A41D 27/20

(57) ABSTRACT

A dual-arm support apparatus comprised of a tubular length of a flexible material that encircles the arms, shoulders, and upper back. Elbows and forearms are comfortably supported in front of the body, and the weight is evenly distributed across the upper back with no pinch or pressure points. Symmetry of design allows for usage inside-out, or upside down. Flexibility of the material allows for non-restrictive arm support in a multitude of positions. The mildly compressive nature of the support may also induce a sense of comfort helping alleviate feelings of anxiety. A pocket may be included to serve as a stuff stack by which the apparatus may be stored when not in use, or to hold a mobile device or wallet during use.

This is a Continuation-in-part of U.S. Design application Ser. No. 29/771,100, filed Feb. 19, 2021. The disclosure of the prior applications is hereby incorporated by reference herein in its entirety.

### BACKGROUND

Related technical fields include arm support devices and travel accessories.

### SUMMARY

Various devices are known that serve to support one or more user's arms. However, each has one or more shortcomings including, but not limited to: being designed for a single arm only; having immobilized or limited arm placement; supporting of only the forearm or elbow, not both; concentrated localization of weight by using straps or thin supports; uncomfortable distribution or location of the weight (e.g., on a back of the user's neck); required attachment to larger body covering or accessory (e.g. a poncho or neck pillow); construction being bulky and unable to fit into a purse or backpack; including buckles or straps that are complex to adjust; unable to be easily and quickly adorned or removed; unable to relieve the weight of the arms from the shoulders or back.

When maintaining an upright seated position for extended periods of time without supportive placement of ones' arms, one may experience associative pain or fatigue in the areas of the neck, shoulders, and upper back. This pain/fatigue may be exacerbated when one tries to support an item in front of them like an electronic device or a book.

Exemplary embodiments of the broad inventive principles described herein provide non-restrictive support of users arms in a variety of positions while comfortably distributing the weight evenly across your upper back. Exemplary support both arms crossed in front of the user, support of one of said arms extended in front of user such as when scrolling through a mobile device, or support of both arms in front of user such as when reading a book.

### BRIEF DESCRIPTION OF THE DRAWINGS

Exemplary embodiments will be described with respect to the following drawings in which:

FIG. 1 is a front, left-side, top perspective view of the dual-arm retention sling in use;

FIG. 2 is a front, top perspective view thereof;

FIG. 3 is a front elevational view thereof;

FIG. 4 is a rear elevational view thereof;

FIG. 5 is a right-side elevational view thereof;

FIG. 6 is a top plan view thereof;

FIG. 7 is a front elevational view thereof, in use;

FIG. 8 is a rear elevational view thereof, in use;

FIG. 9 is a left-side elevational view thereof, in use;

FIG. 10 is a right-side elevational view thereof, in use; and

FIG. 11 is a top plan view thereof, in use.

### DETAILED DESCRIPTION OF EXEMPLARY EMBODIMENTS

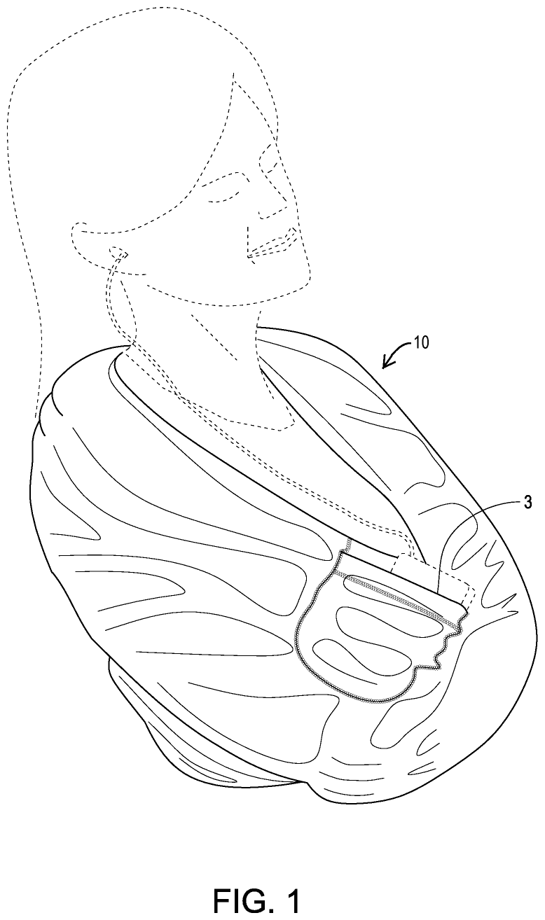

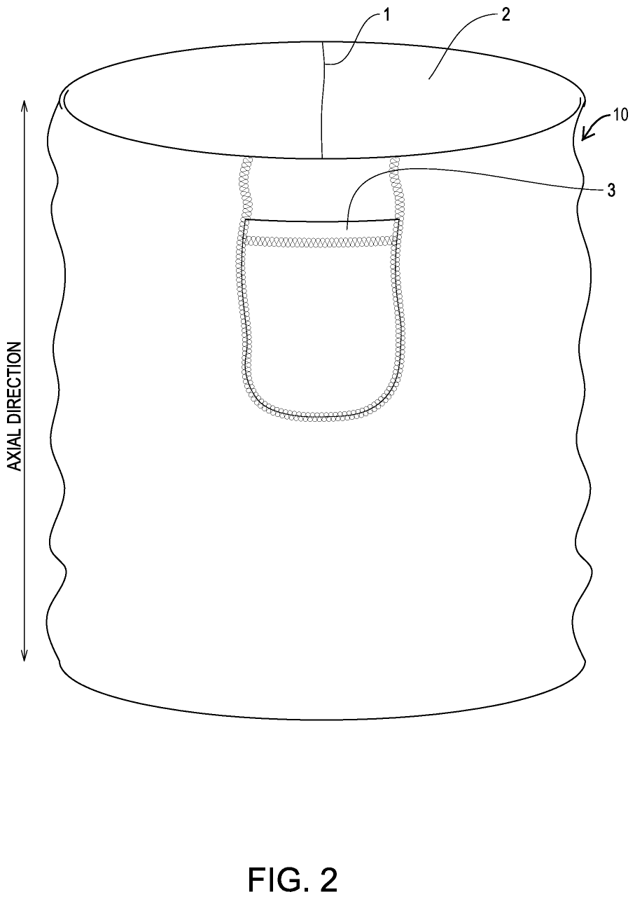

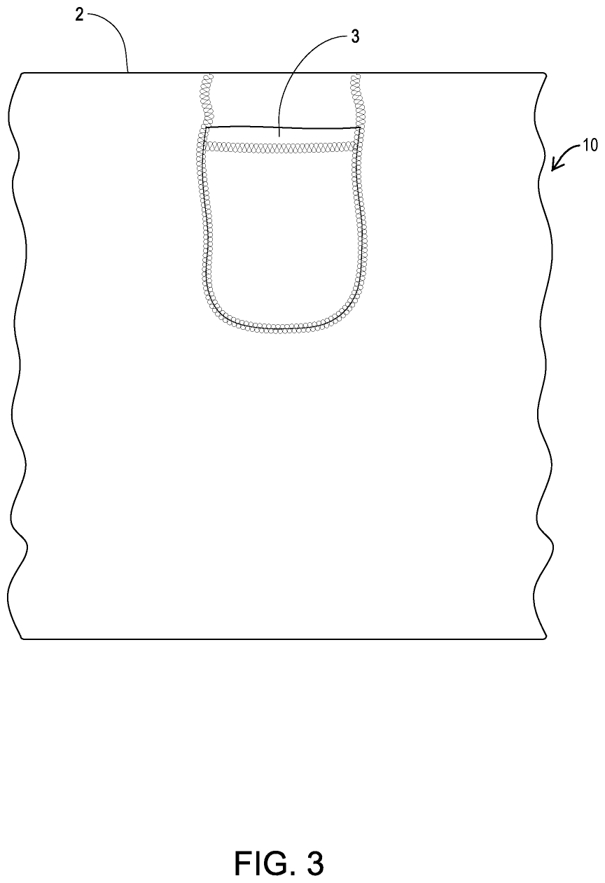

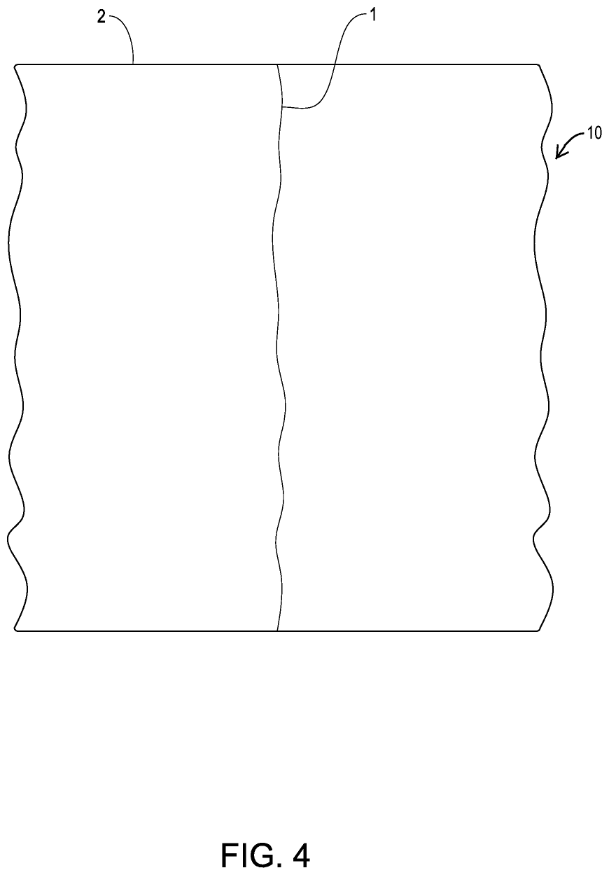

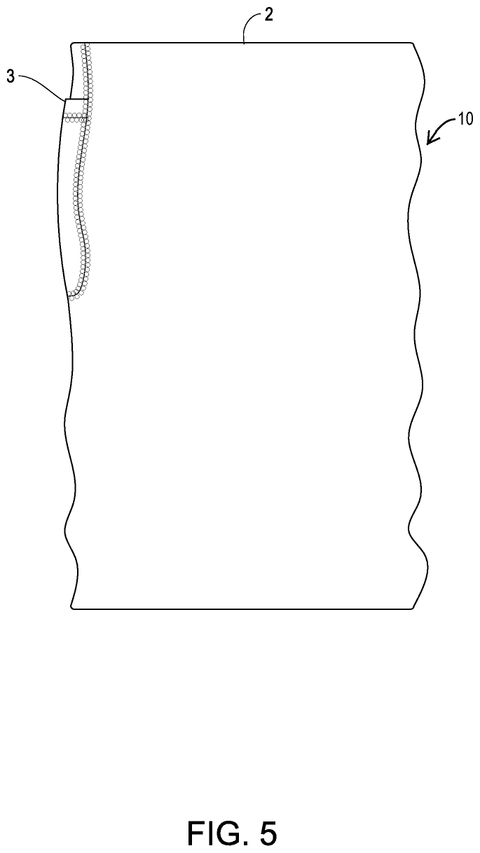

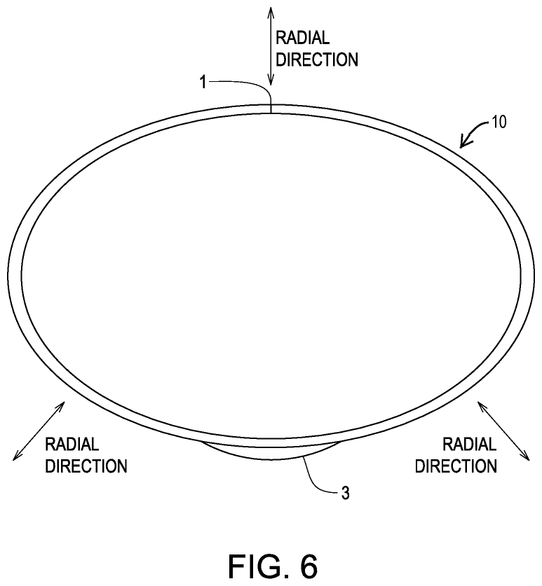

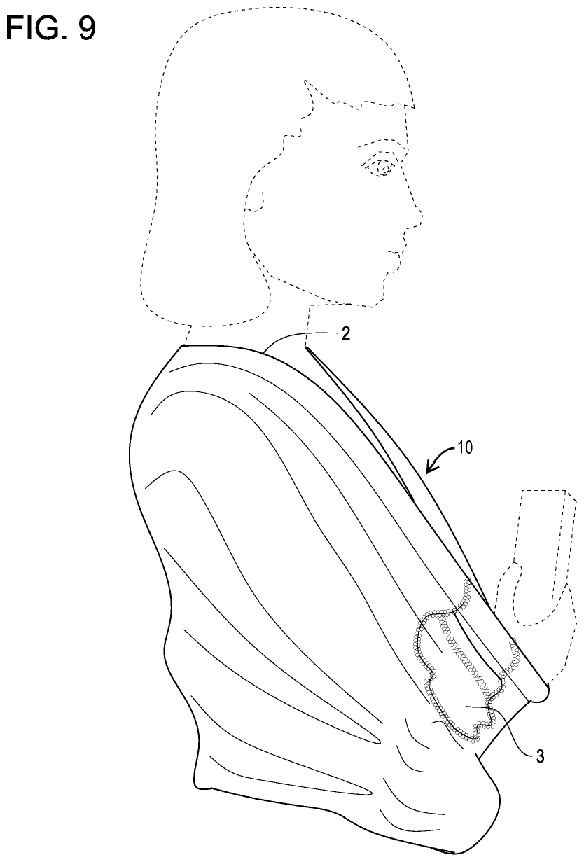

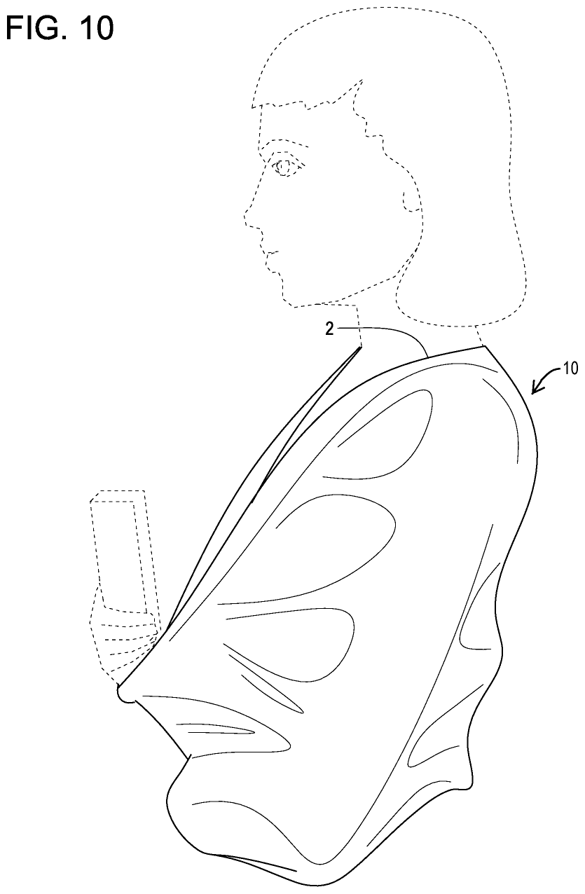

An exemplary embodiment of a dual-arm sling (10) is shown in FIGS. 1-10. As shown in FIG. 2, the sling (10) may take the form of a cylindrical tube. The tube may be made of flexible material that stretches significantly. For example, the tube may stretch significantly in the radial direction (see FIG. 6), with nominal stretch in the axial direction, (see FIG. 2). An example of significant stretch is stretch percentage between 40% to 60%, and preferably between 45% and 55%. An example of nominal stretch is stretch percentage between 0% to 20%, and preferably between 5% and 15%. This type of stretch can be achieved by constructing the tube of the sling (10) from a single or double layer fabric having one stretch percentage on grain and another stretch percentage cross-grain. Significant radial stretch translates into a mild inward compressive force that works with the limited axial stretch to wrap and support the users arms. In addition to providing support, the compressive force may induce a sense of comfort and safety, and mitigate or reduce feelings of anxiety.

The tube of the sling (10) can be constructed of different sizes to accommodate sizes of different people. The proper size of the sling (10) for a user should allow the sling (10) to fit snugly around the torso like that of a comfortable t-shirt. For example a user should select a size of sling (10) so that the radius of the tube in between 95% and 125% of the radius of the user's torso, and preferably between 105% and 115% of the radius of the users torso. However, using a higher percentage of stretch in the radial direction of the sling (10) can allow for more variation between the radius of the user's torso and the radius of the tube.

FIG. 2 shows that the cylindrical tube of the sling (10) can include a vertical seam (1) arranged parallel to an axial direction of the cylindrical tube, and a circumferential seam (2) to allow for a dual-layer non-loop-loom textile construction. Using vertical seam (1) to join the loop minimizes the impedance of stretch because it is perpendicular to the direction of significant stretch in the loop. Placing circumferential seam (2) on a top or bottom edge also minimizes the impedance of stretch in an area where the user's arms will be located (see FIG. 1) and minimizes chafing. The seams can be flat-lock seams or any other type of seam that lays flat to minimize chafing and pressure points. Either the vertical seam (1) or circumferential seam (2) can be omitted by using an endless loop of fabric in the construction of the tube.

An integral pocket (3) may also be included to act as a stuff-sack for storage while not in use, or as a pocket for a phone or wallet while in-use. For example the pocket (3) may store the cylindrical tube in a folded, rolled, or bunched state. The pocket (3) may optionally include a closure mechanism, such as a zipper, button, snap, Velcro, etc. The placement of the pocket (3) ensures that such closure mechanism will not create a pressure point or cause chafing when the sling (10) is worn. The pocket (3) is optional and can be omitted to simplify construction.

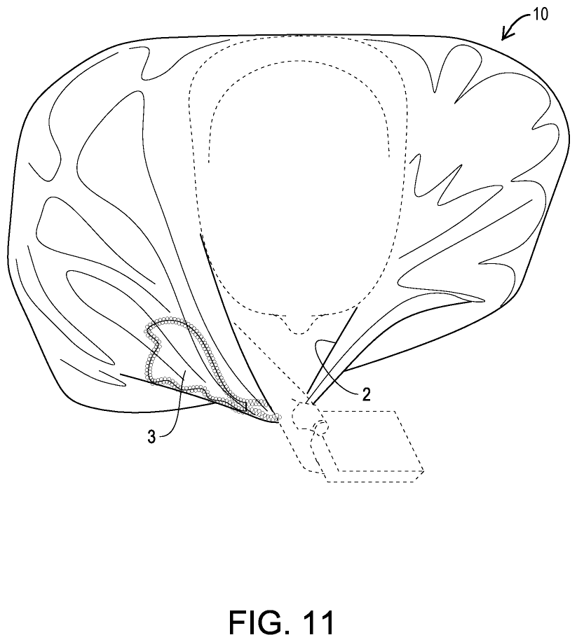

When adorning the sling (10), a user places the sling (10) around the neck similar to an infinity scarf. Then using the opposite arm, one reaches across and wraps invention over the shoulder and elbow. Then the user uses the encapsulated arm to reach across and repeat the process for the other arm. When adorned in this way, the top of the sling (10) covers bother the top of the user's upper back and the top of the user's shoulders as can be seen in FIG. 1 and FIGS. 7-11. Because of its' unique flexible tube structure, adorning or removing the sling (10) requires roughly the same space and agility of putting on a sweater and can be done without assistance or the need for unfastening or fastening any closure devices such as, for example, magnets, snaps, buttons, zippers, clips, buckles, Velcro, etc. Thus, it is possible to adorn and remove the sling (10) without assistance in relatively tight spaces such as when seated on an airplane, on a train, on a bus, or in a car. Also because of its' unique flexible tube structure and lack of closure mechanisms, it is possible to wear the sling (10) comfortably while seated because there are no straps, closure mechanisms, or excessive seams between the user's back and the seat or chair.

FIG. 1 shows an example of the supportive cradling of the arms provided by the flexible tube structure. As shown the arms are comfortably located in front of the user, with the sling (10) wrapping underneath the elbows and forearms, and then up and around the shoulders and upper back. The fabric wraps around the arms in a manner in which the arms are supported by conveyance of the compressive force inward and slightly upward.

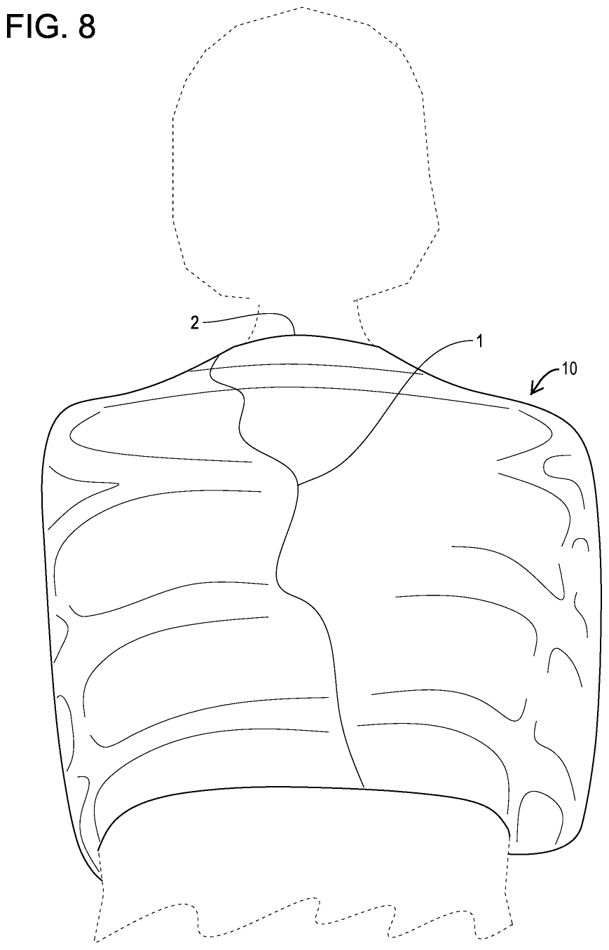

When arms are placed inside the sling (10) the arms are supported by the compressive nature of the sling (10) with the weight of the users arms comfortably supported and distributed evenly across the upper back. FIG. 8 is a rear elevational view showing how the invention covers the upper shoulders and upper back. The weight of the supported arms in front is evenly transferred to the back with no localized concentration of the weight or pinch points caused by straps, closure devices, non-flat seams, or too many seams.

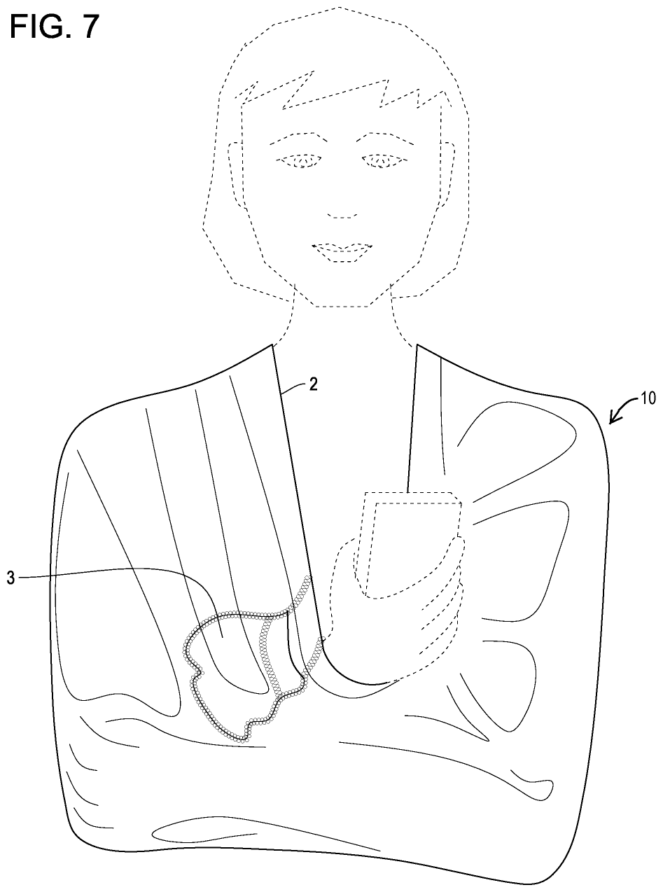

Due to its construction and flexibility, as shown in FIGS. 7-10, an arm (or both arms) may be easily extended in front of user while wearing the sling (10). This allows the user to hold an electronic device or a book in front of them with one or both hands with at least part of the arm(s) remaining supported by the sling (10). For example, FIGS. 7-10 show how the sling (10) maintains support of user's elbow and forearm of an am that is partially exposed to maintain the position of a device in front of user.

As used herein, the term “arm” is meant to encompass the extent of ones' upper extremity including the upper arm, elbow, and forearm. As used herein “flexible material” is meant to encompass either a singular or combination of materials that provide either two-way or four-way stretch and can include, for example, lycra, lycra blends, elastane, elastane blends, nylon, and nylon blends. This flexible material may be single or multiple plies, and may be woven in flat sheets or on a circular loom. As used herein “in front of” is meant to encompass placement either directly adjacent to the body such as when you cross your arms, or when extended outwardly away from the body such as when reading a book.

Although the invention has been described in detail with reference to the exemplary embodiments and variations, additional variations and modifications can be made within the spirit of the broad inventive principles described herein.

Finally, it is noted that, as used in this specification and the appended claims, the singular forms “a”, “an”, and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A dual-arm sling, comprising:
a cylindrical tube constructed of a flexible material, the cylindrical tube being configured to:
be worn by a user so that an inner surface of the cylindrical tube contacts a user's upper back and shoulders; and
provide compressive force on one or both of the user's arms,
wherein the combination of the contact of the inner surface with the user's upper back and shoulders and the compressive force, supports one or both of the user's arms in front of the user's body.

2. The dual-arm sling of claim 1, further comprising a vertical seam arranged parallel to an in an axial direction of the cylindrical tube.

3. The dual-arm sling of claim 1, further comprising a circumferential seam located on one of a top edge or a bottom edge of the cylindrical tube.

4. The dual-arm sling of claim 1, wherein the cylindrical tube is flexible in a radial direction of the cylindrical tube and is flexible in an axial direction of the cylindrical tube.

5. The dual-arm sling of claim 4, wherein a stretch percentage in the radial direction is between 40% to 60%.

6. The dual-arm sling of claim 5, wherein a stretch percentage in the radial direction is between 45% to 55%.

7. The dual-arm sling of claim 4, wherein a stretch percentage in the axial direction is between 0% to 20%.

8. The dual-arm sling of claim 7, wherein a stretch percentage in the axial direction is between 5% and 15%.

9. The dual-arm sling of claim 1, further comprising a pocket.

10. The dual-arm sling of claim 9, wherein the pocket is configured to store the cylindrical tube in a folded, rolled, or bunched state.

11. The dual-arm sling of claim 9, wherein the pocket is configured to store a mobile phone or wallet.

12. The dual-arm sling of claim 9, wherein the pocket comprises a closure mechanism.
